(19) United States
(12) Patent Application Publication
(10) Pub. No.: US 2015/0172681 A1
(43) Pub. Date: Jun. 18, 2015

(54) MEDICAL IMAGE COMPRESSION SYSTEM AND METHOD USING VISUALLY LOSSLESS COMPRESSION

(71) Applicant: SNU R&DB Foundation, Seoul (KR)

(72) Inventors: Kil Joong Kim, Seoul (KR); Bo Hyoung Kim, Seoul (KR); Kyoung Ho Lee, Seongnam (KR)

(73) Assignee: SNU R&DB Foundation

(21) Appl. No.: 14/505,132

(22) Filed: Oct. 2, 2014

Related U.S. Application Data
(63) Continuation of application No. PCT/KR2013/002681, filed on Apr. 1, 2013.

(30) Foreign Application Priority Data
Apr. 2, 2012 (KR) 10-2012-0033713

(51) Int. Cl. H04N 19/196; G06F 19/00; A61B 6/00

(52) U.S. Cl. CPC H04N 19/197; A61B 6/5211; A61B 6/563; G06F 19/321

(57) ABSTRACT

The present invention relates to a medical image compression system and method suitable for visually lossless compression and, more specifically, to a medical image compression system and method capable of obtaining medical images at a high enough compression ratio while preventing loss in information needed to diagnose. The medical image compression system according to one example of the present invention comprises: a storage unit for storing initial learning data and an equation regarding an optimal compression ratio to compress a medical image to be diagnosed at an optimal ratio; a processor for obtaining an optimal compression ratio of the medical image to be diagnosed by using the stored initial learning data and the equation for the optimal compression ratio; and for compressing the medical image to be diagnosed at the optimal compression ratio obtained by the computation unit.

### CROSS-REFERENCE TO RELATED APPLICATION

This application is a continuation of PCT/KR2013/002681 filed on Apr. 1, 2013, which claims priority to Korean Application No. 10-2012-0033713 filed Apr. 2, 2012, which is incorporated herein by reference.

### TECHNICAL FIELD

The present invention relates to an image compression system and method and, more particularly, to an image compression scheme suitable for lossy medical image compression with adjusted compression ratio while preventing diagnostic information from being lost.

### BACKGROUND ART

Technology for compressing medical image data is based on Digital Imaging and Communication in Medicine (DICOM), which is an international standard. In general, image compression is classified into lossy compression and lossless compression depending on whether image data has been lost after restoration. The Joint Photographic Experts Group (JPEG) and Moving Picture Experts Group (MPEG) techniques based on DCT, which is a representative transform coding technique, correspond to lossy compression. Although these techniques support high compression ratios, the justification thereof has not been proved from a medical viewpoint due to concern about misdiagnosis. Accordingly, these techniques are not actually applied to medical diagnosis and treatment. In contrast, lossless compression imparts no concern about misdiagnosis because it does not damage data. That is, if a medical image used in a medical field is stored or transmitted without compression when it is used in a Picture Archiving & Communication System (PACS) or telemedicine, a problem arises in that a large storage space is occupied and transmission is inefficient. If a medical image is compressed and stored or transmitted, a storage space can be reduced and the transmission rate can be improved, but a problem arises in that the important information of an affected area is lost and thus diagnosis and treatment are significantly influenced because of the characteristics of a medical image. Since a medical image used to diagnose a disease requires high image quality due to its specialized use, a lossless compression method having no loss is preferred to lossy compression having a high compression ratio. Accordingly, currently, the PACSs of most hospitals are using lossless compression.

Meanwhile, there is an obligation to store medical image data for a specific period of time because the data may be used in the diagnosis and treatment of a patient in the future. With the development of medical imaging equipment, such as a Magnetic Resonance Imaging (MRI) scanner, a Computed Tomography (CT) scanner, an ultrasonograph, etc., the amount of medical image data is rapidly increasing. Accordingly, the need for the image compression of medical image data is continuously presented. Recently, a method for optimizing the compression ratio of lossy compression so that medical image data is lossy compressed at an appropriate ratio and then stored starts to be proposed.

FIG. 1 is a flowchart of a method for optimizing a lossy compression ratio in accordance with a conventional example.

In this example, a medical image may be compressed at ratio A {A1, A2, . . . , An, n=1, 2, . . . }, and the selection of the ratio is performed by a radiologist. First, a medical image is lossy compressed at ratio A1 at step S101, a radiologist checks the compressed medical image with the naked eye at step S102, and the relevance of the loss ratio is judged at step S103. If the loss ratio is relevant, the ratio A1 is judged to be relevant at step S104, and the lossy compressed image is stored at step S105. If the radiologist judges the loss ratio not to be relevant, the ratio A1 is adjusted, and the image is lossy compressed at a different ratio lower than the ratio A1. In this case, the relevant loss ratio means that although the image has been lossy compressed, a loss is not detectable by the naked eye. In contrast, the irrelevant loss ratio means that the loss ratio of the image is excessively large and thus an original image has been damaged, with the result that the image cannot be used for the diagnosis and treatment of a disease.

The above-described method has the problem of requiring a long period of time because the process of repeatedly adjusting the compression ratio, compressing the medical image, evaluating the medical image with the naked eye of a radiologist, and readjusting the compression ratio should be performed. Furthermore, the above-described method has the problem of causing inconvenience regarding the determination of individual compression ratios because an optimized compression ratio varies with each region of the body or each piece of medical equipment.

Meanwhile, in order to reduce the efforts to reduce the time it takes to store and transmit a medical image, a preceding technology related to a technique for differentially compressing medical images has been presented. For example, Korean Patent Application Publication No. 10-2001-0097394 entitled “Method for differentially compressing medical images” discloses a technique for differentially compressing a medical image having an affected region and a medical image having no affected region. This preceding technology discloses a technology that recognizes an image, distinguishes a portion having an affected region and a portion having no affected region, and performs compression by applying lossless compression to the portion having an affected region and lossy compression to the portion having no affected region.

However, a process of recognizing a portion having an affected region in a medical image is not mentioned in detail in the preceding technology document. Since a portion having an affected region in a medical image varies with each slice, it is difficult to obtain the portion, with the result that the possibility of the overall process of compressing and transmitting a medical image becoming complicated is strong. Furthermore, since the possibility of a radiologist intervening in a process of filtering out an image including an affected region is strong, a user (radiologist) is inconvenienced by an increase in work.

Meanwhile, Korean Patent No. 10-0300955 entitled “Method of Compressing and Restoring Medical Image having Area of Interest” discloses a technology of applying different compression techniques or different compression ratios to an area of interest and an area of non-interest. However, distinguishing the portions of a medical image and applying different compression techniques or compression ratios has the problem of causing the distortion of the image. In this preceding technology document, the details of a method of extracting an area of interest are not described. If a user (radiologist) should define an area of interest for each medical image, the loss of time required for the definition will be considerable.

As a result, there arises the demand for technology for determining an optimum compression ratio, which is capable of ensuring a visually lossless state at a level at which a user can easily use it and there is no concern about misdiagnosis.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

Accordingly, the present invention has been made keeping in mind the above problems occurring in the prior art, and an object of the present invention is to provide a medical image compression system and method that are capable of acquiring medical images having a high compression ratio while preventing diagnostic information from being lost.

In order to achieve the above object, an example of the present invention provides a medical image compression system, including a storage unit configured to store initial learning data used to compress a medical image, acquired by medical equipment and to be examined, at an optimum ratio and a formula related to the optimum compression ratio; a processor configured to obtain the optimum compression ratio for the medical image to be examined using the initial learning data and the formula related to the optimum compression ratio stored in the storage unit; and configured to compress the medical image to be examined at the obtained optimum compression ratio, wherein the obtained optimum compression ratio is obtained for the medical image to be examined to be compressed visually lossless.

The initial learning data and the formula related to the optimum compression ratio and its coefficients may be obtained using a multiple logistic regression (MLR) technique and an Artificial Neural Network (ANN) technique.

The initial learning data may include at least one type of data among patient data, diagnostic data (including comments), information about a diagnostic target organ, order data, and clinician or radiologist information, which are related to the medical image. When computing the optimum compression ratio for the medical image after the formula and the coefficients have been determined, the image compression system and method of the present invention may extract at least one type of data among patient data, diagnostic data, order data, and clinician or radiologist information from information stored in the DICOM header of the corresponding medical image, and then may compute the optimum compression ratio.

The initial learning data may further include information about the medical image's own characteristics in addition to the patient data and the diagnostic data. For example, the initial learning data may further include at least one type of information among the field of view, section thickness and effective tube current-time product of the medical image.

Another example of the present invention provides a medical image compression method executed in a computing system having a storage system and a processor, comprising: storing, in the storage device, initial learning data used to compress a medical image, acquired by medical equipment and to be examined, at an optimum ratio and a formula related to the optimum compression ratio; obtaining, by the processor, the optimum compression ratio for the medical image to be examined using the initial learning data and the formula related to the stored optimum compression ratio; and compressing, by the processor, the medical image to be examined at the obtained optimum compression ratio, wherein the obtained optimum compression ratio is obtained for the medical image to be examined to be compressed visually lossless.

As described above, in accordance with the present invention, a radiologist does not need to evaluate a compressed medical image with the naked eye and then readjust a compression ratio, and thus the time it takes to compress the medical image can be reduced accordingly. That is, by extracting the correlations among the information about the medical equipment, the patient information and other information, and the optimum compression ratio, the need to determine optimized compression ratios for each bodily region of the patient and each piece of medical equipment is eliminated, thereby offering convenience.

Furthermore, a lossy compression ratio for the medical image can be optimized by the medical image compression system, and thus the medical image can be visually lossless compressed, thereby acquiring a medical image having a high compression ratio while preventing diagnostic information from being lost, and thus achieving the advantage of eliminating concern about misdiagnosis upon examination

Moreover, since the size (volume) of a compressed medical image is considerably smaller than an original medical image, the time it takes to transmit the medical image can be reduced, a loss of data within the medical image cannot be visually detected compared to a loss of data of a lossless compressed medical image, and a large amount of medical image information can be stored because a compression ratio is high.

The present invention is a technology for utilizing a database related to an optimum compression ratio, once verified by a radiologist with the naked eye, as initial learning data and extending and applying the initial learning data using a machine-learning technique, and thus has the advantage of incorporating a radiologist's skilled opinion, which differs from a general person's discernment, and thus further reducing concern about misdiagnosis.

Furthermore, the present invention is advantageous in that although a radiologist's efforts are involved in the acquisition of initial learning data, the involvement of a radiologist's efforts can be minimized in a later process of extending and applying data.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a flowchart of a method for optimizing a lossy compression ratio in accordance with a conventional example;

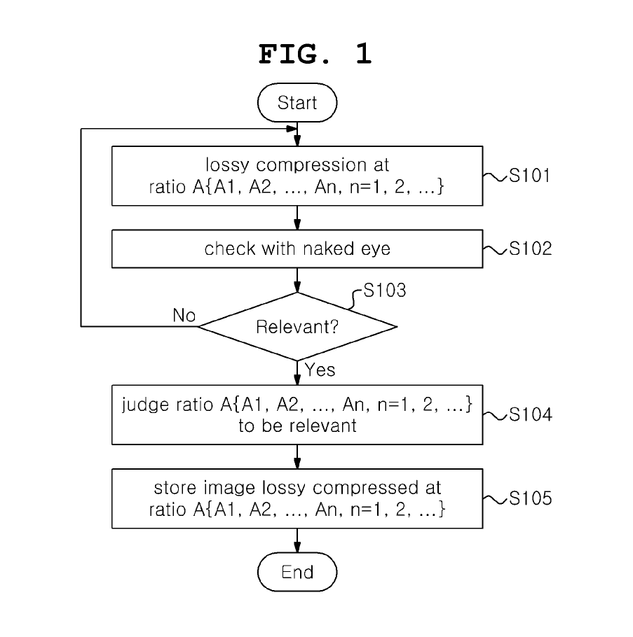

FIG. 2 is a configuration diagram of a medical image compression system using visually lossless compression in accordance with an example of the present invention;

FIG. 3 illustrates an application example of a medical image compression system using visually lossless compression in accordance with an example of the present invention; and

FIG. 4 is a flowchart of a medical image compression method using visually lossless compression in accordance with an example of the present invention.

### DETAILED DESCRIPTION OF THE DISCLOSURE

Hereinafter, preferred embodiments of the present invention will be described in detail with reference to the accompanying drawings. It should be noted that the same reference symbols are assigned to the same components as much as possible even when the components are illustrated in different drawings. In the following description, detailed descriptions of related well-known components or functions that may unnecessarily make the gist of the present invention obscure will be omitted.

<Description of System>

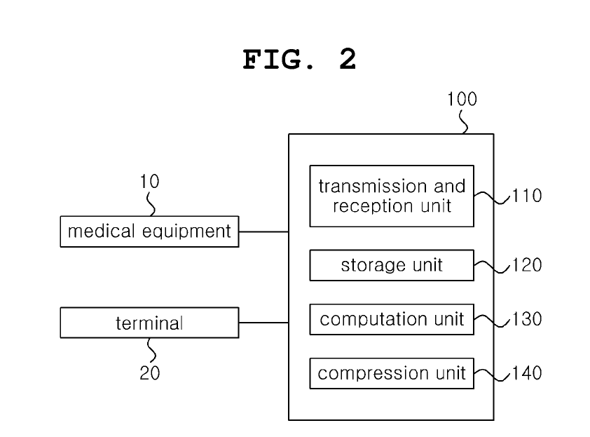

FIG. 2 is a configuration diagram of a medical image compression system using visually lossless compression in accordance with an example of the present invention.

Referring to FIG. 2, the medical image compression system 100 using visually lossless compression in accordance with this example of the present invention includes a transmission and reception unit 110, a storage unit 120, a computation unit 130, and a compression unit 140. The computation unit 130 and the compression unit 140 may be included within a processor (not shown) in the medical image compression system 100.

The transmission and reception unit 110 receives a digitized medical image and information about one of various types of medical equipment 10, such as a Computed Tomography (CT) scanner, a Magnetic Resonance Imaging (MRI) scanner, an endoscope, an ultrasonograph, and the like, from the medical equipment 10, and examination-related physical parameters, that is, medical image information, such as patient information (the name, age, gender, imaged body part and the like of a patient) and imaging technician information corresponding to the medical image, from a terminal 20. In this case, the transmission of the medical image generated by the medical equipment 10 follows the Digital Imaging and Communication in Medicine (DICOM) standard, and old-fashioned medical equipment that does not support the DICOM standard may be provided with additional equipment (not illustrated) that functions to convert medical images into digital form.

The storage unit 120 stores initial learning data required to compress the medical image, acquired from the medical equipment 10 and to be examined, at an optimum compression ratio, and a formula related to the optimum compression ratio. The initial learning data is data including at least one of information about the medical equipment 10, an existing compression ratio for an existing medical image acquired by the medical equipment 10 and existing patient information corresponding to the existing medical image, an existing optimum compression ratio based on the evaluation of the existing medical image by an expert, such as a doctor, and the image characteristic information of the existing medical image. The initial learning data may be used for obtaining coefficients at the optimum compression ratio later by the processor. In greater detail, the existing compression ratio is a compression ratio that is set by medical imaging equipment. The existing optimum compression ratio is basic data about an empirical optimum compression ratio that is determined based on the judgment of the relevance of a loss ratio that is made in such a manner that a doctor (radiologist) or an expert evaluates each compressed image with the naked eye. Coefficients are determined through the above process, and are then stored in the storage unit 120. Meanwhile, according to the example of the invention, the formula related to the optimum compression ratio is characterized in that it is A1X1+A2X2+ . . . +AmXm, where A1, A2, . . . , and Am are the coefficients stored in the storage unit 120, and X1, X2, . . . , and Xm are the information about the medical equipment 10 and the patient information corresponding to the medical image acquired by the medical equipment 10 and to be examined. For example, when the same body parts of two patients are imaged using the same equipment and imaged medical images are compressed at the same compression ratio, one image may be lost and thus there may be concern about misdiagnosis. The reason for this is that although different compression ratios should have been applied because the characteristics of the patients, such as the ages or genders of the patients, were different, patient information has not been taken into account. In the case of the medical equipment 10, it will be apparent that in the same manner, a different compression ratio should be applied depending on the type of medical equipment 10 even when the same body part of a patient is imaged. Accordingly, as described above, it is necessary to extract the correlations between information influencing the compression ratio of the medical image, such as the patient information, the imaged body part and the information about the medical equipment 10, and the optimum compression ratio using the existing compression ratio and then determine coefficients.

Furthermore, the initial learning data includes the image characteristic information of the existing medical image. The image characteristic information includes the degree of the visual recognition visual recognition of the image, and is information representative of the state of the existing medical image.

The initial learning data may be recorded in header information conformable to the DICOM standard, and the storage unit 120 may include and store the initial learning data in DICOM header information that is stored along with medical image data.

The computation unit 130 acquires the optimum compression ratio for the medical image to be examined using the initial learning data and the formula related to the optimum compression ratio stored in the storage unit 120. For this purpose, coefficients are obtained by substituting the existing compression ratio for the optimum compression ratio, and the obtained coefficients may be stored in the storage unit 120.

The computation unit 130 may read patient information, modality (medical imaging equipment, a CT scanner, an MRI scanner, or the like) information, scanning parameters (information required when each piece of medical imaging equipment generates an image), compression ratio (when the medical image has been compressed) and the like from the DICOM header information stored along with the medical image. In addition to this information, the computation unit 130 may read the initial learning data from the DICOM header information.

The computation unit 130 may refer to the existing optimum compression ratio of the existing medical image when determining the optimum compression ratio, and may determine the optimum compression ratio while taking into account the image characteristics (visual characteristics) of the existing medical image.

The initial learning data may be included and stored in the DICOM header information, or may be managed in a file or a database separate from a medical image.

The compression unit 140 compresses the medical image to be examined at the optimum compression ratio obtained by the computation unit 130.

In the present invention, a method of compressing a medical image at an optimum compression ratio is visually lossless compression, neither simple lossy compression nor lossless compression. As described above, simple lossy compression is a compression technique in which a restored image and an original image have a slight mathematical difference, whereas lossless compression is a compression technique in which a restored image is mathematically completely identical to an original image. In contrast, as used in the present invention, the visually lossless (although it is lossy compression in fact) compression refers to a compression technique in which image quality is excellent to such an extent that there is a loss of data of a medical image from a mathematical viewpoint and the loss of data cannot be detected with the naked eye. The visually lossless compression scheme in this invention may present a medical image which has enough information wherewith a radiologist can diagnose faultlessly.

Accordingly, a radiologist does not need to evaluate a compressed medical image with the naked eye and then readjust a compression ratio, and thus the time it takes to compress the medical image can be reduced accordingly. That is, by extracting the correlations among the information about the medical equipment 10, the patient information and other information, and the optimum compression ratio, the need to determine optimized compression ratios for each bodily region of the patient and each piece of medical equipment 10 is eliminated, thereby offering convenience. Furthermore, a lossy compression ratio for the medical image can be optimized by the medical image compression system 100, and thus the medical image can be visually lossless compressed, thereby achieving the advantage of acquiring a medical image having a high compression ratio while preventing diagnostic information from being lost.

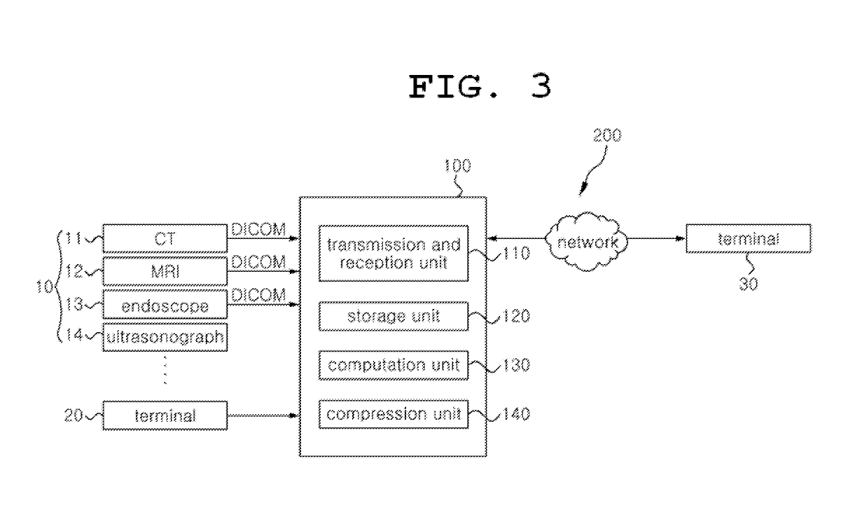

Meanwhile, FIG. 3 illustrates an application example of the medical image compression system using visually lossless compression in accordance with the example of the present invention.

Referring to FIG. 3, the medical image compression system 100 may be utilized under a Picture Archiving & Communication System (PACS) 200 and a telemedicine environment.

The medical image compression system 100 digitizes and stores a medical image imaged by one of various types of medical equipment 10, such as a CT scanner 11, an MRI scanner 12, an endoscope 13, an ultrasonograph 14 and the like, as described above, and the PACS 200 transmits the digitized medical image to the terminal 30 in an examination room or a ward via a network. Accordingly, the medical image for diagnosis and patient treatment is displayed on the terminal 30, and a doctor in charge can view the medical image in real time. Furthermore, the same image can be simultaneously viewed from different locations, various information, such as screen brightness, measurement, enlargement, etc., and convenience are provided, medical personnel required for the management of films can be efficiently relocated, and a medical image can be permanently stored without loss or damage when the medical image is stored. In particular, since the size (volume) of a compressed medical image is considerably smaller than an original medical image, the time it takes to transmit the medical image can be reduced, a loss of data of the medical image cannot be visually detected compared to a loss of data of a lossless compressed medical image, and a large amount of medical image information can be stored because a compression ratio is high. Furthermore, there is no concern about misdiagnosis that may occur when a radiologist or a related doctor makes a diagnosis because the image quality of a restored image is reduced compared to that of an original image.

<Description of Method>

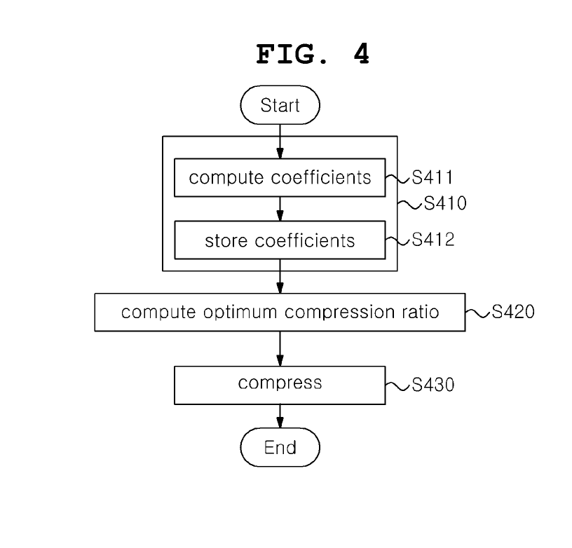

A medical image compression method using visually lossless compression in accordance with an example of the present invention is described with reference to a flowchart illustrated in FIG. 4. The medical image compression method is described in a specific order for ease of description. Descriptions that are the same as the descriptions of the above-described medical image compression system are omitted.

1. Initial Storage Step <S410>

Initial learning data required to compress the medical image, acquired from the medical equipment 10 and to be examined, at an optimum compression ratio, and a formula related to the optimum compression ratio are stored. In this case, the initial learning data includes information about the medical equipment 10, an existing compression ratio for an existing medical image acquired by the medical equipment 10, and existing patient information corresponding to the existing medical image. The existing compression ratio is a basic compression ratio acquired from the medical equipment 10, and an existing optimum compression ratio is an optimum compression ratio that is determined to be optimum by a radiologist through the evaluation of compressed images with the naked eye. The existing patient information refers to the name, age, gender, imaged body part and the like of the patient corresponding to the medical image input from the terminal 20. This information is used as data that is used to obtain coefficients at step S411.

In this case, the data that is used to obtain coefficients may be. A set of data tabulated and stored may use the initial learning data as an independent variable and an optimum compression ratio selected by a medical specialist as a dependent variable. The initial learning data may include at least one type of data among patient data, diagnostic data (including comments), information about a diagnostic target organ, order data, and clinician or radiologist information.

The initial learning data may further include the image characteristics of the medical image and the visual characteristics of the medical image. The visual characteristic of the medical image may be a criterion based on which whether the medical image has been visually lossless compressed is determined.

The initial learning data may further include information about the medical image's own characteristics in addition to the patient data and the diagnostic data. For example, the initial learning data may further include at least one type of information among the field of view, section thickness and effective tube current-time product of the medical image.

A radiologist may participate in a process of establishing a relational database related to the initial learning data and the optimum compression ratio. A radiologist may directly participate in an overall process of establishing a relational database related to the initial learning data and the optimum compression ratio, or may participate in a process of verifying acquired intermediate results.

1-1. Coefficient Computation Step <S411>

Coefficients are obtained by substituting the existing compression ratio into the formula related to the optimum compression ratio. A process of obtaining correlation coefficients from the relational database may be performed using various well-known computation methods or algorithms, such as general linear regression, multiple logistic regression (MLR) and the like.

1-2. Coefficient Storage Step <S412>

The storage unit 120 stores the coefficients obtained at step S411. In this case, the formula related to the optimum compression ratio is characterized in that it is A1X1+A2X2+ . . . +AmXm, where A1, A2, . . . , and Am are the coefficients stored in the storage unit 120, and X1, X2, . . . , and Xm are the information about the medical equipment 10 and the patient information corresponding to the medical image acquired by the medical equipment 10 and to be examined. The acquired correlation coefficients may be stored in a database separate from the relational database, or may be stored in some fields of the relational database.

2. Optimum Compression Ratio Computation Step <S420>

The computation unit 130 acquires the optimum compression ratio for the medical image to be examined using the initial learning data and the optimum compression ratio-related formula stored at step S410.

Step S420 is characterized in that the optimum compression ratio for the medical image to be examined is obtained using the coefficients stored at step S412, the information about the medical equipment 10, and the patient information corresponding to the medical image acquired by the medical equipment 10 and to be examined. After the formula and the coefficients have been determined, at least one type of data among patient data, diagnostic data, order data, and clinician or radiologist information may be extracted from the information stored in the DICOM header of the corresponding medical image, and the optimum compression ratio may be computed, at step S420 of computing the optimum compression ratio for the medical image.

3. Compression Step <S430>

The compression unit 140 compresses the medical image to be examined at the optimum compression ratio obtained at step S420. The compressed image is provided to a radiologist or a related doctor. The loss of data within the image is not detected from a visual viewpoint. Accordingly, a conventional problem in which medical diagnosis and treatment are highly influenced by a loss of important information of an affected part can be overcome, and the effects of minimizing the time it takes to compress an image while compressing the image at an optimum ratio can be achieved.

A medical image compression method in accordance with an example of the present invention may be implemented in the form of program instructions that can be executed by a variety of computer means, and may be stored in a computer-readable storage medium. The computer-readable storage medium may include program instructions, a data file, and a data structure solely or in combination. The program instructions that are stored in the medium may be designed and constructed particularly for the present invention, or may be known and available to those skilled in the field of computer software. Examples of the computer-readable storage medium include magnetic media such as a hard disk, a floppy disk and a magnetic tape, optical media such as CD-ROM and a DVD, magneto-optical media such as a floptical disk, and hardware devices particularly configured to store and execute program instructions such as ROM, RAM, and flash memory. Examples of the program instructions include not only machine language code that is constructed by a compiler but also high-level language code that can be executed by a computer using an interpreter or the like. The above-described hardware components may be configured to act as one or more software modules that perform the operation of the present invention, and vice versa.

While the present invention has been described in conjunction with specific details, such as specific configuration elements, and limited examples and diagrams above, these are provided merely to help an overall understanding of the present invention, the present invention is not limited to these examples, and various modifications and variations can be made from the above description by those having ordinary knowledge in the art to which the present invention pertains.

Accordingly, the technical spirit of the present invention should not be determined based on only the described examples, and the following claims, all equivalent to the claims and equivalent modifications should be construed as falling within the scope of the spirit of the present invention.

The present invention relates to a medical image compression system and method using visually lossless compression and, more particularly, to a medical image compression system and method that are capable of acquiring medical images having a high compression ratio while preventing diagnostic information from being lost.

For this purpose, a medical image compression system in accordance with an example of the present invention includes a storage unit configured to store initial learning data used to compress a medical image, acquired by medical equipment and to be examined, at an optimum ratio and a formula related to the optimum compression ratio; a computation unit configured to obtain the optimum compression ratio for the medical image to be examined using the initial learning data and the formula related to the optimum compression ratio stored in the storage unit; and a compression unit configured to compress the medical image to be examined at the optimum compression ratio obtained by the computation unit.

In accordance with the above configuration, a radiologist does not need to evaluate a compressed medical image with the naked eye and then readjust a compression ratio, and thus the time it takes to compress the medical image can be reduced accordingly. Furthermore, a lossy compression ratio for the medical image can be optimized by the medical image compression system, and thus the medical image can be visually lossless compressed, thereby acquiring a medical image having a high compression ratio while preventing diagnostic information from being lost, and thus achieving the advantage of eliminating concern about misdiagnosis upon examination

What is claimed is:

1. A medical image compression system, comprising:
a storage unit configured to store initial learning data used to compress a medical image, acquired by medical equipment and to be examined, at an optimum ratio and a formula related to the optimum compression ratio;
a processor configured to:
obtain the optimum compression ratio for the medical image to be examined using the initial learning data and the formula related to the optimum compression ratio stored in the storage unit; and
compress the medical image to be examined at the obtained optimum compression ratio, wherein the obtained optimum compression ratio is obtained for the medical image to be examined to be compressed visually lossless.

2. The medical image compression system of claim 1, wherein the initial learning data comprises at least one of information about the medical equipment, an existing compression ratio for an existing medical image acquired by the medical equipment, an existing optimum compression ratio based on evaluation of the existing medical image by an expert, existing patient information corresponding to the existing medical image, and image characteristic information of the existing medical image.

3. The medical image compression system of claim 2, wherein:
the processor is further configured to obtain coefficients by substituting the existing compression ratio or the existing optimum compression ratio into the formula related to the optimum compression ratio; and
the storage unit is further configured to store the coefficients obtained by the computation unit.

4. The medical image compression system of claim 3, wherein the processor is further configured to obtain the optimum compression ratio for the medical image to be examined using at least one of the coefficients stored in the storage unit, the information about the medical equipment, the patient information corresponding to the medical image acquired by the medical equipment and to be examined, the image characteristic information of the medical image, and the existing optimum compression ratio.

5. The medical image compression system of claim 4, wherein the formula related to the optimum compression ratio is A1X1+A2X2+ . . . +AmXm,
where A1, A2, . . . , and Am are the coefficients stored in the storage unit, and X1, X2, . . . , and Xm are the information about the medical equipment and the patient information corresponding to the medical image acquired by the medical equipment and to be examined.

6. A medical image compression method executed in a computing system having a storage device and a processor, comprising:
storing, in the storage device, initial learning data used to compress a medical image, acquired by medical equipment and to be examined, at an optimum ratio and a formula related to the optimum compression ratio;
obtaining, by the processor, the optimum compression ratio for the medical image to be examined using the initial learning data and the formula related to the stored optimum compression ratio; and
compressing, by the processor, the medical image to be examined at the obtained optimum compression ratio, wherein the obtained optimum compression ratio is obtained for the medical image to be examined to be compressed visually lossless.

7. The medical image compression method of claim 6, wherein the initial learning data comprises at least one of information about the medical equipment, an existing compression ratio for an existing medical image acquired by the medical equipment, an existing optimum compression ratio based on evaluation of the existing medical image by an expert, existing patient information corresponding to the existing medical image, and image characteristic information of the existing medical image.

8. The medical image compression method of claim 7, further comprises:
obtaining, by the processor, coefficients by substituting the existing compression ratio or the existing optimum compression ratio into the formula related to the optimum compression ratio; and
storing, in the storage device, the coefficients obtained at the coefficient computation step.

9. The medical image compression method of claim 8, wherein the obtaining the optimum compression ratio comprises:
obtaining, by the processor, the optimum compression ratio for the medical image to be examined using at least one of the coefficients stored at the coefficient storage step, the information about the medical equipment, the patient information corresponding to the medical image acquired by the medical equipment and to be examined, the image characteristic information of the medical image, and the existing optimum compression ratio.

10. The medical image compression method of claim 9, wherein the formula related to the optimum compression ratio is A1X1+A2X2+ . . . +AmXm,
where A1, A2, . . . , and Am are the coefficients stored at the coefficient storage step, and X1, X2, . . . , and Xm are the information about the medical equipment and the patient information corresponding to the medical image acquired by the medical equipment and to be examined.

11. A non-transitory computer-readable storage medium having stored therein program instructions, which when executed by a processor, cause the processor to:
store, in the storage device, initial learning data used to compress a medical image, acquired by medical equipment and to be examined, at an optimum ratio and a formula related to the optimum compression ratio;
obtain the optimum compression ratio for the medical image to be examined using the initial learning data and the formula related to the stored optimum compression ratio; and
compress the medical image to be examined at the obtained optimum compression ratio, wherein the obtained optimum compression ratio is obtained for the medical image to be examined to be compressed visually lossless.
